(19) United States
(12) Patent Application Publication
(10) Pub. No.: US 2010/0092439 A1
(43) Pub. Date: Apr. 15, 2010

(54) ANTIMICROBIALLY-PRIMED MEDICINAL MAGGOT THERAPY

(75) Inventor: Ronald A. Sherman, Irvine, CA (US)

Correspondence Address: GRANT ANDERSON LLP;C/O PORTFOLIOIP, P.O. BOX 52050, MINNEAPOLIS, MN, 55402, US

(73) Assignee: MONARCH LABS LLC, Irvine, CA (US)

(21) Appl. No.: 12/506,158

(22) Filed: Jul. 20, 2009

Related U.S. Application Data
(60) Provisional application No. 61/082,452, filed on Jul. 21, 2008.

(51) Int. Cl. A61K 35/74; A61P 17/02

(52) U.S. Cl. 424/93.42; 424/93.47

(57) ABSTRACT

Antimicrobially-primed medicinal maggots and therapy methods are described for the treatment of a variety of indications including, without limitation, infected or non-healing wounds, soft tissue infection and the like (e.g., gangrenous or infected wound).

### RELATED APPLICATIONS

This application claims priority under 35 USC section 119(e) from U.S. Provisional Application Ser. No. 61/082,452 filed Jul. 21, 2008 by R. Sherman titled “Antimicrobially-Primed Medicinal Maggot Therapy” which is incorporated by reference herein in its entirety.

### FIELD OF THE INVENTION

The invention relates in part to medicinal maggot therapy methods and compositions for treatment of a variety of indications, including, without limitation, wound care, infection and the like (e.g., septic wound care).

### SUMMARY

Maggots utilized for medicinal maggot therapy sometimes do not target one or more microbial strains present in a wound, and infection by such untargeted microbes can persist after maggot therapy. It has been determined that priming a maggot with a priming agent, such as a microbe, an attenuated microbe or a microbial component, for example, and then contacting a wound and/or infection with the primed maggot, may effectively target microbes and reduce the likelihood of post-therapy infection.

Thus, provided in certain embodiments are methods for treating a microbial infection or wound of a subject, which comprises: (a) contacting medicinal maggots with a microbe, an attenuated microbe, a microbial component or similar trigger for the maggot's immunodefense, thereby producing primed maggots; and (b) administering the primed maggots to a subject in need thereof in an amount effective to treat the infection or wound. In certain embodiments, the method comprises separating the microbe, attenuated microbe or microbial component from the medicinal maggots prior to administering the primed maggots to the subject. In some embodiments, a method comprises detecting the presence, absence or amount of an antimicrobial agent produced by the primed maggots prior to administering the primed maggots to the subject.

Also provided in certain embodiments are methods that comprise: (a) contacting medicinal maggots with a microbe associated with an infection or wound; (b) separating the maggots from the microbe after (a), or inactivating the microbe after (a), thereby producing therapeutic maggots; and (c) administering the therapeutic maggots to a subject in need thereof in an amount effective to treat the infection or wound. Also provided, in certain embodiments, are methods for preparing a medicinal maggot for treatment of a microbial infection or wound of a subject, which comprise: contacting medicinal maggots with a microbe, attenuated microbe or microbial component, thereby producing primed maggots. Also, provided in certain embodiments are methods for preparing a medicinal maggot for treatment of a microbial infection or wound of a subject, which comprise: (a) contacting medicinal maggots with a microbe associated with an infection or wound, thereby producing primed maggots, and (b) separating the microbe from the medicinal maggots or inactivating the microbe.

In certain embodiments, a method comprises separating, and removing, the microbe, attenuated microbe or microbial component from the medicinal maggots after priming, and in some embodiments, a method comprises inactivating the microbe or microbial component after priming. In certain embodiments, a method comprises detecting the presence, absence or amount of an antimicrobial agent produced by the primed maggots after they are contacted with the microbe, attenuated microbe or microbial component.

Any suitable method can be utilized to separate a microbe, attenuated microbe or microbial component from a maggot after priming. For example, a composition comprising a maggot and a priming agent may be washed under sterile conditions, and/or filtered, and maggots may be collected. Any suitable method can be utilized to inactivate a microbe, attenuated microbe or microbial component after priming. For example, a composition comprising a maggot and a priming agent can be exposed to (i) sterilization conditions (e.g., ultraviolet radiation, gamma radiation, chemical sterilizer); (ii) an antibiotic that does not kill a significant fraction of maggots in a population (e.g., penicillins, cephalosporins, polymixins, quinolones, sulfonamides, macrolides, tetracyclines, cyclic lipopeptides (e.g., daptomycin), glycylcyclines (e.g., tigecycline), oxazolidinones (e.g., linezolid) and the like), and/or (iii) a bacteriophage that kills or inactivates a microbe but does not kill a significant fraction of maggots in a population (e.g., T4 phage; Caudovirales phage), in some embodiments.

Provided also, in certain embodiments, are compositions comprising a primed medicinal maggot that has been contacted with an attenuated microbe or microbial component. Also provided in some embodiments are compositions comprising a primed medicinal maggot that has been contacted with, and separated from, a pathogenic microbe or microbe associated with an infection or wound. Provided also in certain embodiments is a dressing that comprises primed maggots described herein. Also provided, in some embodiments, is a maggot described herein contained in a container (e.g., plastic container), which maggot often is in substantially sterile form.

Features of embodiments of the invention are described in greater detail in the detailed description and claims that follow.

### DETAILED DESCRIPTION

Larvae of certain blow flies are used therapeutically for wound care. Acknowledged benefits include: debridement (dissolving dead or necrotic tissue), disinfection (killing of bacteria and other microbes), enhanced wound healing (growth stimulation of healthy tissue), and breakdown and inhibition of biofilm. Without being bound by theory, it is expected that pre-exposure to non-pathogenic forms of microbial organisms and/or components thereof (e.g., protein, lipid and/or carbohydrate extracts of bacteria or other germs) stimulate the production of the necessary antimicrobial products by maggots by the time the maggots reach the bedside.

The terms “medicinal maggots” and “medical grade maggots” as used herein refer to insect larvae suitable for medical therapy. Such medicinal maggots generally are capable of (i) debriding necrotic tissue; (ii) disinfecting wounds and (iii) facilitating wound healing. Medicinal maggots are fly larvae, including, without limitation, larvae of the green bottle fly (Phaenicia (Lucilia) sericata). Medicinal maggots are commercially available (e.g., MEDICAL MAGGOTS (Monarch Labs; Irvine, Calif.). Maggots often are provided, and medicinal maggots generally are provided, in substantially sterile form. As used herein, the term “substantially sterile” does not refer to the reproductive state of the maggots, but rather, the environment in which the medicinal maggots are provided. Substantially sterile refers to medicinal maggots, and the conditions under which they are provided, which often are substantially free of one or more of: pathogenic microorganisms, pathogenic viruses, other pathogens, non-pathogenic microorganisms and non-pathogenic viruses. In some embodiments, medicinal maggots and the conditions under which they are provided are substantially free of pathogenic microorganisms, pathogenic viruses and other pathogens.

The term “prime,” and grammatical variants thereof, as used herein refers to contacting a maggot or group of maggots with one or more immunodefense triggering agents. The term “microbe priming agent” as used herein refers to microbes, attenuated microbes and microbe components, and subsets and individual species of the foregoing. The term “primed maggot” as used herein refers to a maggot that has been contacted with the one or more priming agents. Maggots may be primed in any manner in the art that results in an antimicrobial response generated by the maggot.

An antimicrobial response generated by the maggot may be specific for the priming agent used to prime the maggot, or may be broader in spectrum against one or more microbes, attenuated microbes or microbe components other than the priming agent. Thus, a maggot primed with one microbe, attenuated microbe or component from the microbe may generate an antimicrobial response against one or more other microbes, in certain embodiments.

An antimicrobial response can be, or can include, an immune response generated by the maggot against one or more microbes (e.g., generation of antimicrobial peptides by the maggot that disrupt microbe membranes), in some embodiments. Antimicrobial activity of primed maggots can be assessed by one or more antimicrobial activity testing assays known in the art. Examples of antimicrobial assays, include, without limitation, disk or well-diffusion or serial dilution in microbial inhibition or killing assays. Also, assays that determine the presence and/or amount of one or more antimicrobial products can include such techniques as chromatography, electrophoresis, immunostaining and the like. Such methods can be used to determine the presence, absence or level (e.g., potency, amount) of an antimicrobial response or antimicrobial components (e.g., antimicrobial peptides) produced by primed maggots. In certain embodiments, a primed maggot, or group of primed maggots, kills greater than or equal to about 10%, 20%, 30%, 40%, 50%, 60%, 70%, 80%, 85%, 90%, 95% or more of a microbe in an antimicrobial response, and in a time frame suitable for medicinal maggot therapy, as determined by a antimicrobial assay.

The terms “microbe” and “microbial organism” as used herein refer to pathogenic and non-pathogenic microbial organisms. The term “pathogenic microbe” as used herein refers to microbes associated with an infection and/or wound, which include, without limitation, bacteria (e.g., Gram negative bacteria; Gram positive bacteria), yeast, fungi, amoebae, protozoa, viruses and the like. Particular non-limiting examples of bacteria associated with wounds and infection include, without limitation, bacteria such as Pseudomonas sp. (e.g., Pseudomonas aerugenosa); Staphylococcus sp. (e.g., Staphylococcus aureus); group A and B streptococci, and a variety of gram-positive aerobic and anaerobic bacteria. Others include, without limitation, Escherichia sp. (e.g., Escherichia coli), Micrococcus sp. (e.g., Micrococcus luteus), Proteus sp. (e.g., Proteus mirabilis), Mycobacterium sp. (e.g., M. ulcerans), Mucor sp. (e.g., M. plumbeus, M. racemosus), and the like. Examples of protozoa include, without limitation, Leischmania species (e.g., L. major, L. tropica, L. aethiopica, L. mexicana, L. braziliensis, L. donovani, L. infantum). A microbe can be from any convenient source, including, without limitation, from a depository (e.g., American Type Culture Collection; Manassas, Va.), laboratory, health care facility or vehicle, or field (e.g., pathogen outbreak site). In certain embodiments, a microbe is from a wound or infection of a patient, the microbe is used to prime a maggot, and the primed maggot is administered to the same patient from which the microbe was taken (e.g., for individualized and customized treatment), or to another patient in some embodiments.

In certain embodiments, non-pathogenic microbes, or components of such microbes, may be utilized to prime maggots, as such microbes and components can trigger and induce antimicrobial responses in the primed maggots against pathogenic microbes. An advantage of the latter embodiments, for example, is the primed maggots can be administered to a subject without risk of the microbe causing infection should not all of the priming microbe be removed or separated from the maggot before the latter is used for maggot therapy.

Maggots may be contacted with one or more microbes or attenuated microbes in some embodiments. In certain embodiments, a maggot is primed with one, two, three, four, five, six, seven, eight, nine, ten or more different microbes or attenuated microbes. The term “attenuated microbe” as used herein refers to a microbe has been modified such that it is less infectious than its native microbe counterpart, and generates an immune response in a maggot. Attenuation methods are in the art, and include, without limitation, (i) passaging the microbe through a foreign host (e.g., tissue culture, embryonated eggs, live animals), and (ii) killing the microbe to yield an inactivated microbe (e.g., formalin treatment).

When primed with a microbe, a primed maggot may be sanitized with respect to the microbes. For example, after inducing the maggot immune response with the bacteria, a primed maggot may be exposed to a specific viral parasite of that bacterium (e.g., bacteriophage), which would kill the bacteria and then die itself. Other non-limiting examples of inactivating microbes, attenuated microbes and/or microbial components, and/or separating the foregoing from primed maggots, are described herein.

In certain embodiments, maggots may be contacted with one or more microbe components. The term “microbe component” as used herein refers to a component from a microbe that can generate an immune response against the microbe by the exposed maggot. Microbe components sometimes are molecules on the microbe surface in certain embodiments, including, without limitation, peptide, polypeptide, protein, lipid and carbohydrate components. Peptide components, in certain embodiments, can be about 5 to about 200 amino acids in length (e.g., about 10, 20, 30, 40, 50, 60, 70, 80, 90, 100, 110, 120, 130, 140, 150, 160, 170, 180 amino acids in length). Microbe components may be prepared by a variety of methods known in the art, which include without limitation, methods for extracting components from microbes and methods for synthesizing components (e.g., recombinant peptide, polypeptide or protein expression system; synthetic production of peptides). Examples of components that can be used to prime maggots include, without limitation, enterotoxin (e.g., enterotoxin A, enterotoxin B); toxic shock syndrome toxin; protein A; lipoteichoic acid (LTA); peptidoglycan (PG), concanavalin A (Con A); lipopolysaccharide (LPS); endotoxin; lipid (e.g., lipid A); monophosphoryl lipid (e.g., monophosphoryl lipid A); mitogen, ovalbumin (OVA); metalloproteinase (e.g., aureolysin, pseudolysin, bacillolysin); lectin; phytohaemaglutinin (PHA); acemannan and coenzyme Q10. One or more microbe components can be utilized to prime a maggot (e.g., 2, 3, 4, 5, 6, 7, 8, 9, 10 or more components).

Microbe components sometimes are isolated, and in some embodiments, are not isolated. The terms “isolated”, “isolating” or “isolation” as used herein refer to material removed from its original environment (e.g., the natural environment if it is naturally occurring, or a host cell if expressed exogenously), and thus is altered “by the hand of man” from its original environment. The terms “isolated”, “isolating” or “isolation” and “purified”, “purifying” or “purification” as used herein with reference to molecules does not refer to absolute purity. Rather, “purified”, “purifying” or “purification” refers to a substance in a composition that contains fewer substance species in the same class (e.g., nucleic acid or protein species) other than the substance of interest in comparison to the sample from which it originated. “Purified”, “purifying” or “purification”, if a nucleic acid or protein for example, refers to a substance in a composition that contains fewer nucleic acid species or protein species other than the nucleic acid or protein of interest in comparison to the sample from which it originated. In certain embodiments, an isolated microbial component is provided substantially purified form (e.g., about 90%, 91%, 92%, 93%, 94%, 95%, 96%, 97%, 98%, 99% or greater than 99% free of another component). Non-isolated microbial components sometimes are provided in a cell lysate or partially purified cell lysate.

In certain embodiments, maggots may be contacted with one or more substances (for example, molecules, chemicals, peptides, polypeptides, proteins, glycoproteins) not derived from microbes but which are found also to stimulate production of antimicrobial agents by the maggot. Examples of such substances include, without limitation, cytokines, such as for example, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18), interferons (e.g., IFN-beta, IFN-gamma), tumor necrosis factors (e.g., TNF-alpha, TNF-beta), lymphokines, monokines and chemokines; growth factors (e.g., transforming growth factors (e.g., TGF-alpha, TGF-beta)); and the like. Other examples include molecules chemically synthesized or derived such that it has the same or essentially (that is, structurally and/or physiologically) the same characteristics as a isolated microbial molecule.

A maggot may be contacted with one or more priming agents in any order or at the same time (e.g., in one formulation). A maggot may be contacted with a priming agent in a variety of manners. Methods of contacting include, but are not limited to, inoculation, transcutaneous or transovarian transmission, or ingestion. In some embodiments, a priming agent is delivered directly or via a carrier molecule or vector. In certain embodiments, maggot hatchlings (e.g., 1st instars) are fed extracts of one or more types of priming agents, such that by the time maggots arrive for administration to the subject, the maggots are producing antimicrobial agents against microbes in the wound or infection to be treated. A priming agent may be separated from maggots and/or inactivated before administration of primed maggots to a subject. The person of ordinary skill in the art can separate, remove and/or inactivate priming agents from maggots using a variety of methods, including, but not limited to, washing, dissolving, filtering, or centrifuging.

Primed maggots can be administered to a subject in need of a treatment of an infection or wound by the person of ordinary skill in the art. The term “subject” as used herein refers to humans and any animals pertinent for veterinary applications (e.g., ungulate (e.g., equine, bovine, caprine, porcine), canine, feline, avian, reptilian, amphibian, fish). The person of ordinary skill in the art, often a physician or other health care provider, can identify a subject in need of administration of maggots. For example, a non-healing wound with infection, dead (necrotic) or foreign debris is a wound candidate for maggot therapy. A subject with a microbe infection that can be disinfected by a maggot also can be identified by a person of ordinary skill in the art. Infections include, without limitation, infections associated with wounds and with infections of the flesh (e.g., Staphylococcus sp. (e.g., S. aureus), Streptococcus sp., Mycobacterium sp. (e.g., M. ulcerans), Mucor sp., Leischmania sp.). In certain embodiments, infections identified for the treatment by primed medicinal maggots include, without limitation, infections by Staphylococcus bacteria (e.g., Staphylococcus aureus); group A and B streptococci, Gram-positive aerobic and anaerobic bacteria, and gram negative bacteria, such as Pseudomonas bacteria (e.g., Pseudomonas aerugenosa). Primed maggots described herein may be used to treat infections caused by antibiotic resistant microbes, such as methicillin-resistant S. aureus (MRSA), for example. A person of ordinary skill in the art can select the number of maggots for administration that will result in an amount effective to treat a wound and/or infection, and administration methods are known. A commonly recommended dose of medicinal maggots, for example, is 5-10 larvae/sq centimeter of wound surface area.

Maggots described herein may be provided in conjunction with a dressing for use in treatment of a wound and/or infection. Dressings may be of any suitable type, and sometimes (i) form a seal around a wound or infection, to prevent maggots from leaving the wound or infection, and (ii) are air permeable, to provide oxygen to the maggots.

### EXAMPLES

The examples set forth below illustrate certain embodiments and do not limit the invention.

### Example 1

### Methods for Preparing and Testing Primed Medical Grade Maggots

Provided hereafter are protocols for demonstrating that P. sericata produces detectable antimicrobial compounds in response to non-traumatic (i.e., enteric or transcuticular) induction (stimulus).

Preparation of Antigenic Challenge

The following microbial and antigenic suspensions are prepared to determine an appropriate stimulus and its concentration. Once the general protocol is performed, other related or prospective inducers of innate host immunity (IIHU) are similarly tested. Microbial and antigenic IIHU's are prepared as solutions of increasing concentration for testing. Microbial IIHU's are prepared in tryptocase soy broth (TSB) in serial 10-fold dilutions of 0 to 106 colony forming units (CFU) per ml, as measured by optical density and confirmed with a simple plating bioassay on agar plates. Antigenic IIHU's are prepared in sterile water, in concentrations from 0-5% wt/vol (50 mg/ml) in 2× serial dilutions.

Microbial Inducers of Innate Host Immunity (IIHU):

Staphylococcus aureus 502a (ATCC #27217)

Escherichia coli ML35 (ATCC #43827)

Micrococcus luteus (ATCC # 4698)

Pseudomonas aerugenosa QM B1468 (AATCC 147-1982; ATTC #13388)

Pseudomonas aerugenosa FDA Drug testing strain (ATTC #14502)

Proteus mirabilis CDC PR 14 Type strain (ATCC #29906)

Antigen Inducers of Innate Host Immunity (IIHU):

Monophosphoryl Lipid A, Synthetic (Avanti Polar Lipids, Inc)

Staphylococcus enterotoxin A (SEA)

Staphylococcus Protein A

Superantigen Staphylococcus enterotoxin A (SEA)

Superantigen Staphylococcus aureus enterotoxin B (SEB)

Toxic shock syndrome toxin-1 (TSST-1)

Staphylococcus Protein A

Lipoteichoic acid (LTA) from Staphylococcus saprophyticus strain S1

Peptidoglycan (PG)

Concanavalin A (Con A)

Protein A

SAC (Cowan type I Staphylococcus aureus)

Lipopolysaccharides (LPS)

E. coli endotoxin

Monophosphoryl Lipid A (Synthetic E. coli endotoxin)

pokeweed mitogen (PWM)

ovalbumin (OVA)

Thermolysin-like metalloproteinase

Acemannan

coenzyme Q10

Background of Certain Antigen Inducers:

Superantigen Staphylococcus enterotoxin A (SEA) stimulates T cells.

Lipoteichoic acid (LTA) from Staphylococcus saprophyticus strain S1 may induce thymocyte proliferation and maturation in BALB/c-mice after systemic administration.

Nowell (Nowell P C. Phytohemagglutinin: an initiator of mitosis in cultures of normal human leukocytes. Cancer Res. 1960 May; 20:462-6) determined that normal human lymphocytes proliferated into blast forms in vitro in the presence of Phytohaemaglutinin (PHA), a lectin extracted from the red kidney bean Phaseolus vulgaris. Subsequently, proliferative and blastogenic responses of lymphocytes of man and other higher vertebrates, mice and chickens to other mitogens such as Concanavalin A (Con A), Pokeweed mitogen (PWM) and Lipopolysaccharide have been reported (Chessin L N, Börjeson J, Welsh P D, Douglas S D, Cooper H L. Studies on human peripheral blood lymphocytes in vitro. II. Morphological and biochemical studies on the transformation of lymphocytes by pokeweed mitogen. J Exp Med. 1966 Nov. 1; 124 (5):873-84). These observations and the subsequent discoveries of many other forms and sources of mitogens have since been applied by numerous researchers (Jannossy G, Greaves M F. Lymphocyte activation. I. Response of T and B lymphocytes to phytomitogens; Clin Exp Immunol. 1971; 9:483-98; Etlinger H M, Hodgins H O, Chiller J M. J Immunol. 1976 June; 116 (6):1547-53. Evolution of the lymphoid system. I. Evidence for lymphocyte heterogeneity in rainbow trout revealed by the organ distribution of mitogenic responses; Etlinger H M, Hodgins H O, Chiller J M. Evolution of the lymphoid system. III. Morphological and functional consequences of mitogenic stimulation of rainbow trout lymphocytes. Dev Comp Immunol. 1978 April; 2 (2):263-76) to study characteristics of lymphocytes.

Thermolysin-like metalloproteinases such as aureolysin, pseudolysin, and bacillolysin represent virulence factors of diverse bacterial pathogens.

A. vera's beneficial properties may be attributed to mucopolysaccharides present in the inner gel of the leaf, especially acemannan (acetylated mannans). An injectable form of acemannan manufactured and marketed by Carrington Laboratories as Acemannan Immunostimulant™ has been approved in the USA for treatment of fibrosarcoma (a type of cancer) in dogs and cats after clinical trials. It has not been approved for use by humans, and, although it is not a drug, its sale is controlled and it can only be obtained through a veterinary doctor.

Challenge

Phaenicia (Lucilia) sericata eggs are chemically disinfected (Sherman & Wyle, 1996 [Sherman R A and Wyle F A: Low-cost, low-maintenance rearing of maggots in hospitals, clinics, and schools. Am J Trop Med Hyg. 54 (1): 38-41. 1996.]). Groups of 100 disinfected eggs are incubated overnight at 80° F. within a sealed and perforated (pore size=160 um) polyester net bag in sterile covered 100 mm diameter Petri dish with 1.0 ml sterile normal saline and allowed to hatch. Hatchlings then are transferred, within the bag, to a covered Petri dish containing 2 ml of an IIHU solution. For negative controls, 2 ml of sterile water or TSB is used. The larvae stays afloat in the bag and feed/drink from the solution in the dish for 12 hours at “room temperature” (74-80° F.).

The net bag containing larvae is removed with sterile forceps and rinsed 3 times in sterile water. The larvae then are transferred to another sterile Petri dish containing 3 ml sterile soy-based Maggot Media (the media used to transport Medical Maggots) for 12 hours.

Collection of Antimicrobial Secretions

Larvae, about 24 hours old, are transferred to a closed-system ground-glass filter funnel, irrigated with sterile saline (10 ml/hr) at room temperature and fitted with an air vent. Secretions (effluent) are collected by intermittent suction, over ice, and then frozen hourly for a total of 12 hours. Just prior to use, the effluent (approximately 120 ml) are defrosted, pooled, concentrated, and re-suspended in 100 ul 0.01% acetic acid. Bioactive agents are serially diluted to assess relative potency. Protein assay are performed for those samples demonstrating antimicrobial activity.

Bioassay of Antimicrobial Secretions

Antimicrobial activity in the secretions are assessed using two assays.

A) Lawn Overlay Bioassay

Lawn agar is prepared in 9×9 cm square plates by cooling 10 ml of boiled TSA to 45° C. and adding 106 CFU of the assay bacteria. Assay bacteria are growth phase Staphylococcus aureus, Escherichia coli and Pseudomonas aerugenosa, listed above, which are washed, concentrated and re-suspended in approximately 10 ul of 10 mM pipes buffer, pH 7.4. After the lawn solidifies, 25 evenly spaced 3 mm diameter wells are punched out. Secretion samples then are added (see preparation method above), 5 ul to each well. After incubation for 3 hours at 37° C., 10 ml of TSA is added as an “overlay agar.” The assays are incubated overnight at 37° C. and zones (ring) of growth inhibition are recorded.

B) Serial Dilution Bactericidal Assay

The same assay bacteria described in the lawn overlay assay (Staphylococcus aureus, Escherichia coli and Pseudomonas aerugenosa, strains listed above) are used in the bactericidal assay after growing in TSB for 2.5 hours, washing and concentrating the pellets, and re-suspending in 10 mM pipes buffer (pH 7.4) to a concentration of 106 per ml (based on optical density at 620 nm). Ten microliters of the assay bacteria are added to each of 6 test tubes containing increasing concentrations of the test sample in 10 mM Pipes buffer (pH 7.4), totaling 40 ul. This mixture, plus the 10 ul of bacteria, will equal a total of 50 ul of bacterial suspension. After sealing the tubes and incubating at 37° C. for 1 hour, 30 ul from each tube are withdrawn and serially diluted 10-fold, 3 times. Then, 100 ul aliquots from each diluted specimen are spread over a TSA agar plate in duplicate and incubated overnight at 37° C. Bactericidal activity of the test samples are assessed the following morning by counting colonies.

### Example 2

### Testing Primed Maggots for Safety and Efficacy

Once the most potent antigens have been identified, primed larvae are tested for safety and efficacy. The safety of activated maggots is assessed using the standard rat burn model, on which live second instars (larvae) are placed on a 1 cm diameter full-thickness wound surgically cut on the back of rats, applying a dressing of 10 (standard human dose or 100 larvae (that is, 10 times the recommended dose) to the wound. On the other side of the back, a similar wound is made, covering it with a dressing containing no maggots. Larvae are allowed to grow for 3 days before the dressings are removed (typical treatment course in humans is 48-72 hours). The animals are evaluated daily for signs of infection, inflammation or abnormal or delayed wound healing.

### Example 3

### Testing Primed Maggots for Efficacy in Humans

Efficacy studies in humans are performed in standard “prospective clinical trial” fashion, for pressure ulcers and diabetic foot ulcers. Methods of application, data collection and analysis are described in detail, as part of earlier maggot therapy clinical trials (Sherman et al, 1995 [Sherman R A, Wyle F, and Vulpe M: Maggot Debridement Therapy for treating pressure ulcers in spinal cord injury patients. J Spinal Cord Med, 18 (2): 71-74. 1995.]; Sherman, 2002 [Sherman R A: Maggot vs conservative debridement therapy for the treatment of pressure ulcers. Wound Repair and Regeneration. 2002; 10:208-14.]; Sherman, 2003 [Sherman R A: Cohort study of maggot therapy for treating diabetic foot ulcers. Diabetes Care 2003; 26 (2):446-51]; Markevich et al, 1998 [Markevich Y O, McLeod-Roberts J, Mousley M, Melloy E. Maggot therapy for diabetic neuropathic foot wounds: a randomized study. European Association for the Study of Diabetes 17-21 Sep. 2000, Jerusalem, Israel: 2000 (Abstract)]).

### Example 4

### Examples of Certain Embodiments

Non-limiting examples of certain embodiments of the invention follow.
1. A method for treating a microbial infection or wound of a subject, which comprises:
   a. contacting medicinal maggots with one or more priming agents, wherein the priming agents are selected from the group consisting of microbes, attenuated microbes or microbial components, thereby producing primed maggots; and
   b. administering the primed maggots to a subject in need thereof in an amount effective to treat the infection or wound.
2. The method of embodiment 1, which comprises separating the priming agents from the medicinal maggots prior to (b).
3. The method of embodiment 1, which comprises detecting the presence, absence or amount of antimicrobial agents or activity produced by the primed maggots prior to (b).
4. The method of embodiment 1, wherein the subject is human.
5. The method of embodiment 1, wherein the agent is a microbe.
6. The method of embodiment 5, wherein a microbe is a bacterium.
7. The method of embodiment 6, wherein the microbe is selected from the group consisting of a Staphylococcus bacterium, Staphylococcus aureus, Escherichia bacterium, Escherichia coli, Micrococcus bacterium, Micrococcus luteus, Pseudomonas bacterium, Pseudomonas aerugenosa, a Proteus bacterium, and Proteus mirabilis.
8. The method of embodiment 7, wherein the bacterium is a Pseudomonas bacterium.
9. The method of embodiment 7, wherein the Pseudomonas bacterium is Pseudomonas aerugenosa.
10. The method of embodiment 1, wherein the agent is an attenuated microbe.
11. Then method of embodiment 1, wherein the agent is a microbial component.
12. The method of embodiment 11, wherein the microbial component is selected from the group consisting of enterotoxin; toxic shock syndrome toxin; protein A; lipoteichoic acid (LTA); peptidoglycan (PG), concanavalin A (Con A); lipopolysaccharide (LPS); endotoxin; lipid; monophosphoryl lipid; mitogen, ovalbumin (OVA); metalloproteinase; lectin; phytohaemaglutinin (PHA); acemannan and coenzyme Q10.
13. A method for preparing a medicinal maggot for treatment of a microbial infection or wound of a subject, which comprises contacting medicinal maggots with an attenuated microbe or isolated microbial component, thereby producing primed maggots.
14. The method of embodiment 13, which comprises separating the attenuated microbe or microbial component from the medicinal maggots.
15. The method of embodiment 13, which comprises detecting the presence, absence or amount of an antimicrobial agent produced by the primed maggots after they are contacted with the attenuated microbe or microbial component.
16. A composition comprising a medicinal maggot that has been contacted with an attenuated microbe or microbial component.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, yet these modifications and improvements are within the scope and spirit of the invention.

The invention illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the invention claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” is about 1, about 2 and about 3). For example, a weight of “about 100 grams” can include weights between 90 grams and 110 grams. Thus, it should be understood that although the present invention has been specifically disclosed by representative embodiments and optional features, modification and variation of the concepts herein disclosed may be resorted to by those skilled in the art, and such modifications and variations are considered within the scope of this invention. Certain examples of embodiments of the invention are set forth in the claims that follow.

What is claimed is:

1. A method for treating a microbial infection or wound of a subject, which comprises:
a. contacting medicinal maggots with one or more priming agents, wherein the priming agents are selected from the group consisting of microbes, attenuated microbes or microbial components, thereby producing primed maggots; and
b. administering the primed maggots to a subject in need thereof in an amount effective to treat the infection or wound.

2. The method of claim 1, which comprises separating the priming agents from the medicinal maggots prior to (b).

3. The method of claim 1, which comprises detecting the presence, absence or amount of antimicrobial agents or activity produced by the primed maggots prior to (b).

4. The method of claim 1, wherein the subject is human.

5. The method of claim 1, wherein the agent is a microbe.

6. The method of claim 5, wherein a microbe is a bacterium.

7. The method of claim 6, wherein the microbe is selected from the group consisting of a Staphylococcus bacterium, Staphylococcus aureus, Escherichia bacterium, Escherichia coli, Micrococcus bacterium, Micrococcus luteus, Pseudomonas bacterium, Pseudomonas aerugenosa, a Proteus bacterium, and Proteus mirabilis.

8. The method of claim 7, wherein the bacterium is a Pseudomonas baceterium.

9. The method of claim 7, wherein the Pseudomonas bacterium is Pseudomonas aerugenosa.

10. The method of claim 1, wherein the agent is an attenuated microbe.

11. Then method of claim 1, wherein the agent is a microbial component.

12. The method of claim 11, wherein the microbial component is selected from the group consisting of enterotoxin; toxic shock syndrome toxin; protein A; lipoteichoic acid (LTA); peptidoglycan (PG), concanavalin A (Con A); lipopolysaccharide (LPS); endotoxin; monophosphoryl lipid; mitogen, ovalbumin (OVA); metalloproteinase; lectin; phytohaemaglutinin (PHA); acemannan and coenzyme Q10.

13. A method for preparing a medicinal maggot for treatment of a microbial infection or wound of a subject, which comprises contacting medicinal maggots with an attenuated microbe or microbial component, thereby producing primed maggots.

14. The method of claim 13, which comprises separating the attenuated microbe or microbial component from the medicinal maggots.

15. The method of claim 13, which comprises detecting the presence, absence or amount of an antimicrobial agent produced by the primed maggots after they are contacted with the attenuated microbe or microbial component.

16. A composition comprising a medicinal maggot that has been contacted with an attenuated microbe or microbial component.
